(19) United States
(12) Patent Application Publication
(10) Pub. No.: US 2005/0119514 A1
(43) Pub. Date: Jun. 2, 2005

(54) Oligomerization of olefins comprising at least three carbon atoms

(75) Inventors: Shahram Mihan, Ludwigshafen (DE); Zheiko Maas, Mannheim (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/506,602

(22) PCT Filed: Mar. 11, 2003
(86) PCT No.: PCT/EP03/02495

(30) Foreign Application Priority Data
Mar. 14, 2002 (DE) 102 11 373.4

(51) Int. Cl. C07C 2/02

(52) U.S. Cl. 585/521

(57) ABSTRACT

A description is given of a process for the oligomerization of α-olefins having at least 3 carbon atoms, in which the olefin is brought into contact with a catalyst system obtainable from a chromium source, a 1,3,5-trialkyl-1,3,5-triazacyclohexane, where the alkyl radical has no α, β or γ branching, and at least one activator comprising a boron compound, with the molar ratio B:Cr being at least 5. The process allows the preparation of trimers of the α-olefin in high yield and with high selectivity.

The present invention relates to a process for the oligomerization of α-olefins having at least three carbon atoms.

Olefin oligomers having up to 30 carbon atoms have great economic importance as comonomers for plastics or as precursors of oxo alcohols which are in turn a constituent of surfactants and plasticizers for plastics. Processes for the oligomerization of lower olefins which come, for example, from steam crackers are thus of central importance for the production of products which are part of daily life.

WO 00/58319 describes a process for preparing oligomers of olefins using an oligomerization catalyst which is obtainable from a chromium compound and a 1,3,5-triazacyclohexane plus an activating additive. According to WO 00/58319, the radicals on the nitrogen atoms of the triazacyclohexane are preferably β-alkyl-substituted alkyl, for example 2-ethylhexyl or 2-n-propylheptyl. Preferred olefins for the oligomerization are olefins having from 2 to 10 carbon atoms. The examples of WO 00/58319 describe the oligomerization of ethene and of 1-butene. In all examples in which 1-butene is used, use is made of a catalyst in which the radicals on the nitrogen atoms of the triazacyclohexane have β-alkyl branching. According to WO 00/58319, a boron compound and an aluminum alkyl can be used as activating additive.

It is an object of the present invention to provide a process of the type mentioned at the outset which leads to high yields of olefin oligomers and preferably gives defined oligomers, in particular trimers, with high selectivity.

We have found that this object is achieved by a process for the oligomerization of α-olefins having at least three carbon atoms, in which the olefin is brought into contact with a catalyst system obtainable from
a) at least one chromium source,
b) at least one ligand of the formula I 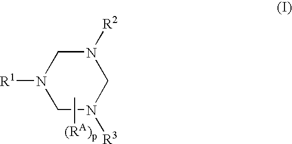
where R¹ to R³ are each, independently of one another, C₄-C₃₀-alkyl which has no α, β or γ branching,
R^{A} is an organic group having from 1 to 30 carbon atoms which is bound via a silicon atom or a carbon atom, and
   p is from 0 to 6, preferably from 0 to 3, and
c) at least one activator comprising a boron compound, with the molar ratio of B:Cr being at least 5.

The radicals R¹ to R³ are each, independently of one another, C₄-C₃₀-alkyl which has no α, β or γ branching, but they can be branched at a position further removed from the nitrogen atom at which they are bound. R¹ to R³ are preferably each, independently of one another, linear C₄-C₃₀-alkyl, more preferably linear C₄-C₁₈-alkyl such as n-butyl, n-hexyl, n-octyl or n-dodecyl.

R^{A} is, for example, C₁-C₁₈-alkyl, preferably C₁-C₁₂-alkyl such as methyl, ethyl, n-propyl, i-propyl, butyl, pentyl, hexyl; C₅-C₇-cycloalkyl such as cyclopentyl and cyclohexyl; C₆-C₁₅-aryl such as phenyl, methylphenyl or naphthyl; or C₇-C₁₅-arylalkyl such as benzyl. Possible radicals R^{A} bound via a silicon atom are, for example, trialkylsilyl groups such as trimethylsilyl. When p is 3, the radicals R^{A} are preferably arranged symmetrically, i.e. each carbon atom in the triazacyclohexane ring bears a radical R^{A}.

Preferred ligands of the formula I are

1,3,5-tri-n-dodecyl-1,3,5-triazacyclohexane,

1,3,5-tri-n-octyl-1,3,5-triazacyclohexane,

1,3,5-tri-n-hexyl-1,3,5-triazacyclohexane and

1,3,5-tri-n-butyl-1,3,5-triazacyclohexane.

The ligands of the formula I in which p is 0 and the radicals R¹ to R³ are identical can be prepared in a manner known per se, in particular by reacting primary amines with formaldehyde or paraformaldehyde. Analogously, ligands of the formula I which bear radicals R^{A} on the carbon atoms of the triazacyclohexane ring can be obtained from primary amines and appropriate aldehydes and/or ketones. For suitable methods of preparation, reference may be made to WO 00/58319 and the references cited therein. The processes described can be employed analogously for the preparation of the ligands of the formula I.

Suitable chromium sources are chromium(II) and/or preferably chromium(III) compounds. Suitable chromium(III) compounds are, in particular, those of the-formula CrX₃, where X is an abstractable counterion, in particular halogen such as fluorine, bromine, iodine, in particular chlorine, tosylate, triflate, tetrafluoroborate, hexafluorophosphate, hexafluoroantimonate, tetraphenylborate; C₁-C₁₈-carboxylate such as acetate, butyrate, neopentanoate, laurate, stearate or 2-ethylhexanoate. CrCl₃ has been found to be particularly useful.

Further suitable chromium sources are compounds of the formula CrX₃L₃, where X is as defined above and L is an uncharged complexing ligand, e.g. ether complexes such as CrCl₃(tetrahydrofuran)₃, CrCl₃(dioxane)₃, ester complexes such as CrCl₃(n-butylacetate), CrCl₃(ethylacetate), alcohol complexes such as CrCl₃(i-propanol)₃, CrCl₃(2-ethylhexanol)₃, amine complexes such as CrCl₃(pyridine)₃, CrCl₃(i-propylamine)₃ or nitrile complexes such as CrCl₃(acetonitrile)₃.

It is possible to prepare a chromium complex from the chromium source and the ligand of the formula I by methods known per se (e.g., for example, W. A. Herrmann, A. Salzer: “Synthetic Methods of Organometallic and Anorganic Chemistry”, Vol. 1, Thieme-Verlag Stuttgart, 1996) and to isolate this and use it in the process of the present invention. As an alternative, the chromium source and the ligand of the formula I are brought into contact in situ in the reaction medium. The ligand of the formula I is generally used in an at least equimolar amount based on the chromium source (calculated as chromium atoms).

According to the present invention, an activator comprising at least one boron compound is also used. The boron compound is used in such an amount that the molar ratio B:Cr is at least 5, preferably from 10 to 100. Examples of suitable boron compounds are compounds of the formula BZ₃ and/or Cat^{⊕}BZ₄^{⊖}, where Z is an electron-withdrawing radical, preferably a phenyl radical which bears from 2 to 5 substituents selected from among fluorine atoms, trifluoromethyl and pentafluoroethyl, and Cat^{⊕} is a cation, preferably a tertiary or quaternary ammonium cation or a triarylcarbonium ion. Preferred radicals Z are pentafluorophenyl and 3,5-bisperfluoromethylphenyl. Suitable cations cat^{⊕} are, for example, tritylium, tri(C₁-C₈-alkyl)ammonium, tetra(C₁-C₈-alkyl)ammonium, di(C₁-C₄-alkyl)anilinium, di(C₁-C₄-alkyl)benzylammonium and the like.

Preferred boron compounds are selected from among trispentafluorophenylborane, N,N-dimethylanilinium tetrakispentafluorophenylborate, tri-n-butylammonium tetrakispentafluorophenylborate, N,N-dimethylanilinium tetrakis(3,5-bisperfluoromethylphenyl)borate, tri-n-butylammonium tetrakis(3,5-bisperfluoromethylphenyl)borate and tritylium tetrakispentafluorophenylborate. Among these, particular preference is given to tritylium tetrakispentafluorophenylborate, trispentafluorophenylborane and, in particular, dimethylanilinium tetrakis(pentafluorophenyl)borate.

Apart from the boron compound, the activator generally further comprises a metal compound having at least one metal-carbon bond. For the purposes of the present patent application, such compounds are referred to collectively as “metal alkyl compounds”. Representative metal alkyl compounds are alkylaluminum compounds, alkylmagnesium compounds, alkylzinc compounds and/or alkyllithium compounds. Among these, preference is given to alkylaluminum compounds. They can have the formulae AlR₃, AlR₂Hal, AlRHal₂, AlR₂OR′, AlRHalOR′ or Al₂R₃Hal₃, where R and R′ are each, independently of one another, methyl, ethyl or a straight-chain or branched C₃-C₈-alkyl group and R′ may also be aryl such as phenyl and Hal is a halogen atom such as fluorine, bromine, iodine or in particular chlorine. Representative compounds are trimethylaluminum, triethylaluminum, tri-n-propylaluminum, triisopropylaluminum, tri-n-butylaluminum, triisobutylaluminum, diethylaluminum chloride, diethylaluminum bromide, diethylaluminum ethoxide, diethylaluminum phenoxide and ethylaluminum ethoxide fluoride.

In preferred embodiments, the activator comprises a trialkylaluminum such as trimethylaluminum, triethylaluminum, tri-n-propylaluminum, triisopropylaluminum, tri-n-butylaluminum or triisobutylaluminum and an alkylaluminum halide such as diethylaluminum chloride, ethylaluminum dichloride or diethylaluminum bromide. The molar ratio of trialkylaluminum to alkylaluminum halide is preferably 1-50:1, in particular 3-20:1.

The molar ratio of chromium source to metal alkyl compound, in particular alkylaluminum compound, is usually from 1:1 to 1:200, preferably from 1:5 to 1:100.

The process of the present invention is generally carried out in the liquid phase in a solvent. Suitable solvents include aprotic solvents, e.g. aliphatic saturated hydrocarbons such as butane, pentane, 3-methylpentane, hexane, heptane, 2-methylhexane, octane, cyclohexane, methylcyclohexane, 2,2,4-trimethylpentane, decalin; halogenated hydrocarbons such as dichloroethane; aromatic hydrocarbons such as benzene, toluene, xylene, ethylbenzene, mesitylene or tetralin.

The process of the present invention is suitable for the oligomerization, in particular the selective trimerization, of α-olefins having from at least 3 to preferably 10 carbon atoms, e.g. 1-propene, 1-butene, 1-hexene or 1-decene. A particularly suitable olefin is 1-butene, if desired in admixture with its isomers as are present, for instance, in raffinate II.

Because of the tendency of the metal alkyl compounds used as activators to hydrolyze, the process of the present invention is generally carried out with substantial exclusion of moisture. It is preferably carried out under protective gas. Protective gases which can be used are all gases which are chemically inert under the reaction conditions, e.g. nitrogen or argon. The olefin to be reacted can itself also take over the function of the protective gas, as long as it has a sufficiently high vapor pressure under the reaction conditions.

The oligomerization is preferably carried out at from 0 to 120° C., in particular from 25 to 110° C. It is generally carried out at a pressure from ambient pressure to 120° C.

After the reaction is complete, the catalyst system is generally deactivated. Suitable deactivators are, for example, water, which may, if desired, be acidified, and lower alcohols. The products of the oligomerization are advantageously purified by distillation. Unreacted starting material can be recovered and returned to the reaction.

The invention is illustrated by the following examples.

### EXAMPLES

The amount indicated in the table below of [(1,3,5-tri-n-dodecyl-1,3,5-triazacyclohexane)CrCl₃] (Examples 1-3) or [(l,3,5-tris(2-ethylhexyl)-1,3,5-triazacyclohexane)CrCl₃] (Example 4, corresponds to Example 21 of WO 00/58319) together with 250 ml of toluene were placed in a 1 L four-necked flask provided with contact thermometer, stirrer, heating mantle and gas inlet tube at 40° C. under argon. The amount indicated in the table of dimethylanilinium tetrakis(pentafluorophenyl)borate (DMAB) was added, the reaction mixture was heated to 70° C., cooled to 40° C. and the indicated amounts of triisobutylaluminum (TIBAL) and diethylaluminum chloride (DEAC) were added.

1-Butene was passed through the resulting light-green/yellow solution. The temperature was maintained at 40° C. After one hour, the reaction was stopped by addition of 15 ml of concentrated hydrochloric acid in 50 ml of methanol and the reaction mixture was stirred for another 15 minutes. 250 ml of methanol were then added and the mixture was stirred for a further 15 minutes. After filtration, the product was washed three times with water and dried over sodium sulfate. The yield of dodecene was determined by gas-chromatographic analysis of the resulting solution.

TABLE

| | | | | | Activity |
|---|---|---|---|---|---|
| Ex. | Cat [μmol] | DMAB* | TIBAL** | DEAC** | kgC₁₂/mol_{Cr}/h |
| 1 | 39.4 | 10 | 50 | 5 | 283 |
| 2 | 38.5 | 10 | 50 | — | 175 |
| 3*** | 40.6 | 2 | 50 | — | 130 |
| 4*** | 38.1 | 10 | 50 | — | 67 |

*Molar ratio of B:Cr
**Molar ratio of Al:Cr
***Comparative examples

1: A process for the oligomerization of α-olefins having at least three carbon atoms, in which the olefin is brought into contact with a catalyst system obtainable from
a) at least one chromium source,
b) at least one ligand of the formula I 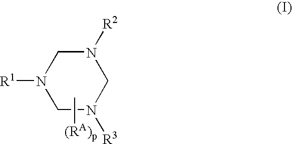
where R¹ to R³ are each, independently of one another, C₄-C₃₀-alkyl which has no α, β or γ branching,
R^{a} is an organic group having from 1 to 30 carbon atoms which is bound via a silicon atom or a carbon atom, and
p is from 0 to 6, and
c) at least one activator comprising a boron compound, with the molar ratio of B:Cr being at least 5.

2: A process as claimed in claim 1, wherein the activator further comprises an alkylaluminum compound.

3: A process as claimed in claim 2, wherein the activator comprises a trialkylaluminum and an alkylaluminum halide.

4: A process as claimed in claim 1, wherein 1,3,5-tri-n-dodecyl-1,3,5-triazacyclohexane is used as ligand.

5: A process as claimed in claim 1, wherein the boron compound has the formula BZ₃ and/or Cat^{?} BZ₄^{?}, where Z is an electron-withdrawing radical and Cat^{?} is a cation.

6: A process as claimed in claim 5, wherein the boron compound is selected from the group consisting of trispentafluorophenylborane, N,N-dimethylanilinium tetrakispentafluorophenylborate, tri-n-butylammonium tetrakispentafluorophenylborate, N,N-dimethylanilinium tetrakis(3,5-bisperfluoromethylphenyl)borate, tri-n-butylammonium tetrakis(3,5-bisperfluoromethylphenyl)borate and tritylium tetrakispentafluorophenylborate and mixtures thereof.

7: A process as claimed in claim 1, wherein 1-butene is used as olefin.
